(19) United States
(12) Patent Application Publication
(10) Pub. No.: US 2022/0378256 A1
(43) Pub. Date: Dec. 1, 2022

(54) Portable hand sanitizing system in a fanny pack

(71) Applicant: Simon Bordeleau, Levis (CA)

(72) Inventor: Simon Bordeleau, Levis (CA)

(21) Appl. No.: 17/331,373

(22) Filed: May 26, 2021

(51) Int. Cl. A47K 5/12; A61L 2/00; A45F 3/00

(52) U.S. Cl. CPC A47K 5/1201; A61L 2/0088; A45F 3/005; A61L 2202/16; A61L 2202/17; A61L 2202/181

(57) ABSTRACT

A fanny pack or small pack that has at least one but preferably two or more pockets made out of an absorbent lining material capable of holding a quantity of a sanitizing substance imbibed therein.

### CROSS-REFERENCE TO RELATED APPLICATIONS

The present application claims priority to number GB2007939.8xxx, filed on May 27, 2020 entitled “Portable hand sanitizing system”, the disclosure of which is hereby incorporated in its entirety at least by reference.

### BACKGROUND OF THE INVENTION

### 1. Field of the Invention

The present invention relates generally to hygiene and sanitation equipment, but more particularly to a portable and wearable hand sanitizing system.

### 2. Description of Related Art

The new normal brought about the COVID-19 pandemic has people made more aware of hand washing and other hygienic precautions. This is even more so in some occupations beyond healthcare workers. As with anything, the easier a task is made the more people will do it. Access to hand washing cleaning products is crucial for that.

### BRIEF SUMMARY OF THE INVENTION

It is a main object of the present disclosure to provide for a portable and wearable hand sanitizing system.

In order to do so, there is provided a container such as a fanny pack or small pack that has at least one but preferably two or more pockets made out of an absorbent lining material capable of holding a quantity of a sanitizing substance imbibed therein. Each pocket is made out of a durable liquid proof impermeable material, and the interior lining of the pocket s made out of an absorbent material having anti-bacterial and anti-viral properties. There are fibers made out of copper which have shown such properties, as a non-limiting example. There are many ways by which the disinfectant keep its properties for extended periods of time inside the pockets.

The pockets are reversible so as to facilitate their washing and drying and are further comprised of an impermeable lining so as to keep the sanitizing substance within the absorbent lining material

There is a color coded pull out tab for opening a flap which exposes one of the two pockets, and a pull back tab to close the flap, the two tabs, being of different colors so as to prevent the user from contaminating his hands. A backside portion of the flap having a scrubbing surface to clean fingernails.

Hook and loop patches are used for securing the flaps in either a closed or open configuration.

A backside portion of the flap having a scrubbing surface to clean fingernails.

Hook and loop patches are used for securing the flaps in either a closed or open configuration.

The fanny pack has belt loop and thigh straps to attach to a user.

The fanny pack has a ring, attached to the fanny pack for carrying a dry towel.

The portable hand sanitizing system has a method of use consisting in the steps of:

a) a user first filling the pockets with the sanitizing substance.

b) the user fills the pockets slowly to ensure that the absorbent lining material absorbs the sanitizing substance and that the liquid does not spill out of the pockets.

c) the user pulls with his finger on a pull out tab which is color coded to open a flap which exposes one of the two pockets. A backside portion of the flap having a scrubbing surface to clean fingernails.

d) the user then puts his hand inside the pocket which is moist with the sanitizing substance which is held captive by way of the absorbent lining material.

e) after use, the user grasps a pull back tab to close the flap, the pull tab and the pull back tab, being of different colors, preventing the user from contaminating his hands by having the user not touching the pull out tab after having washed his hands.

f) the user then repeats the process with his other hand, using the other pocket.

### BRIEF DESCRIPTION OF THE SEVERAL VIEWS OF THE DRAWINGS

Other features and advantages of the present invention will become apparent when the following detailed description is read in conjunction with the accompanying drawings, in which:

FIGS. 1A-B are an isometric views of an embodiment of the invention with flaps open and closed, respectively, according to an embodiment of the invention.

FIG. 2A-B Side and front view of an embodiment of the invention.

FIGS. 3A-B Front views of users using the two embodiments of the invention.

### DETAILED DESCRIPTION OF THE PREFERRED EMBODIMENT

The following description is provided to enable any person skilled in the art to make and use the invention and sets forth the best modes contemplated by the inventor of carrying out his invention. Various modifications, however, will remain readily apparent to those skilled in the art, since the general principles of the present invention have been defined herein.

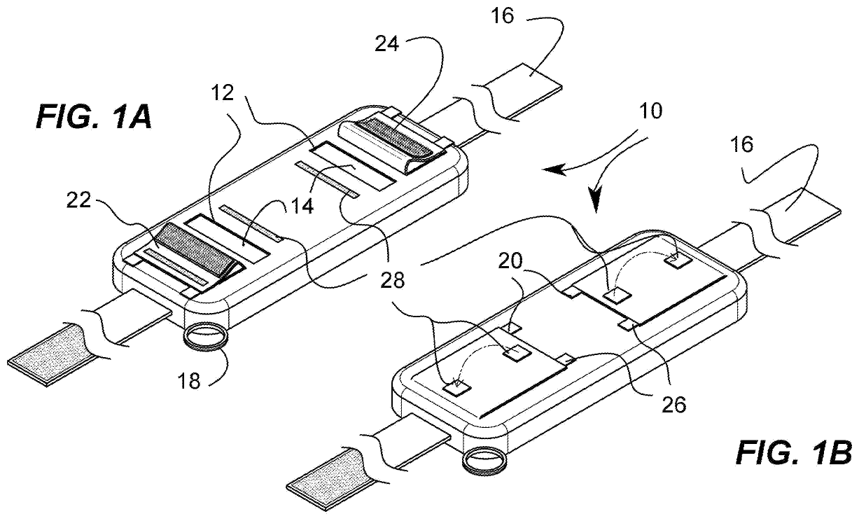

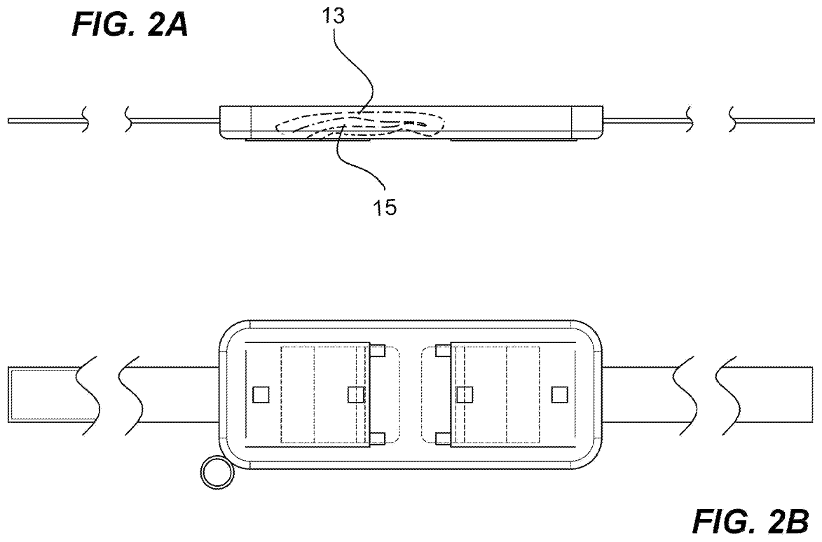

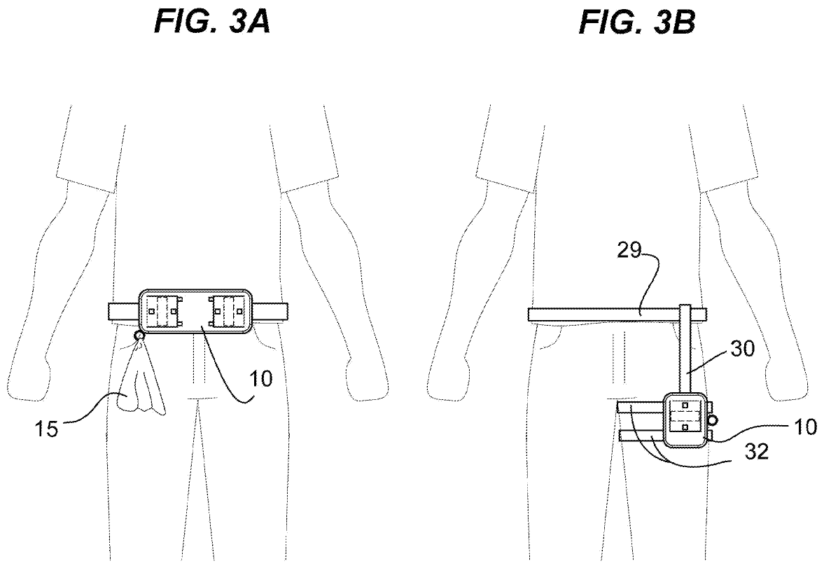

Referring now to any of FIGS. 1-3, there is provided a fanny pack (10) that can hold at least one but preferably two or more pockets (12) having an interior made out of an absorbent lining material (15) which can absorb a quantity of a sanitizing substance (14) so as to prevent that liquid from simply spilling out of the pocket (12).

One embodiment has a belt designed to strap (16) around the waist of the intended user, as known in the art, while another embodiment attaches to an existing belt (29) with a belt attachment loop (30) and thigh straps (32). In both embodiments, a ring (18) can be used if one wants to carry a dry towel (15).

The method of use consists in the steps of a user first filling the pockets (12) with the sanitizing substance (14) which can be selected from the group comprising alcohol based soap, for example, which can come in liquid or gel form. The user fills the pockets (12) slowly to ensure that the absorbent lining material (15) absorbs the sanitizing substance (4) so that it does not spill out of the pockets (12).

In use, the user pulls with his finger on a pull out tab (20) which is color coded (EX: Red) to open a flap (22) which exposes one of the two pockets (12). A backside portion of the flap (22) has a scrubbing surface (24) to clean fingernails. The user then puts his hand inside the pocket (12) which is moist with the sanitizing substance (14) which is held captive by way of the absorbent lining material (15) which can hold and keep the sanitizing substance (14) for repeated usage throughout the day. After use, the user grasps a pull back tab (26) to close the flap (22). The two tabs (20, 26) which are of different colors (EX: Red, Green) prevent the user from contaminating his hands by having the user not touching the pull out tab (20) after having washed his hands. The user then repeats the process with his other hand, using the other pocket (12). Various hook and loop patches (28) help in securing the flaps (22) in either a closed or open configuration.

The fanny pack (10) has reversible pockets (12), e.g. they can be pulled inside out like pulling pockets out of pants. This facilitates their washing and drying, which makes the full pack (10) easily washable, preferably by hand wash. The pockets (12) have an outer impermeable lining (13) so as to keep the sanitizing substance (14) within the absorbent lining material (15). The absorbent lining material (15) is generally sponge-like so as to release enough of the sanitizing substance (14) when a user puts his hand inside the pocket (12).

Although the invention has been described in considerable detail in language specific to structural features, it is to be understood that the invention defined in the appended claims is not necessarily limited to the specific features described. Rather, the specific features are disclosed as exemplary preferred forms of implementing the claimed invention. Stated otherwise, it is to be understood that the phraseology and terminology employed herein, as well as the abstract, are for the purpose of description and should not be regarded as limiting. Therefore, while exemplary illustrative embodiments of the invention have been described, numerous variations and alternative embodiments will occur to those skilled in the art. Such variations and alternate embodiments are contemplated, and can be made without departing from the spirit and scope of the invention.

It should further be noted that throughout the entire disclosure, the labels such as left, right, front, back, top, bottom, forward, reverse, clockwise, counter clockwise, up, down, or other similar terms such as upper, lower, aft, fore, vertical, horizontal, oblique, proximal, distal, parallel, perpendicular, transverse, longitudinal, etc. have been used for convenience purposes only and are not intended to imply any particular fixed direction or orientation. Instead, they are used to reflect relative locations and/or directions/orientations between various portions of an object.

In addition, reference to “first,” “second,” “third,” and etc. members throughout the disclosure (and in particular, claims) are not used to show a serial or numerical limitation but instead are used to distinguish or identify the various members of the group.

1. A portable hand sanitizing system in the form of a fanny pack with comprising: at least one but preferably two or more pockets made out of an absorbent lining material capable of holding a quantity of a sanitizing substance imbibed therein.

2. The portable hand sanitizing system of claim 1 wherein the pockets are reversible so as to facilitate their washing and drying.

3. The portable hand sanitizing system of claim 1 wherein the pockets are further comprised of an impermeable lining so as to keep the sanitizing substance within the absorbent lining material

4. The portable hand sanitizing system of claim 1 wherein: there is a color coded pull out tab for opening a flap which exposes one of the two pockets, and a pull back tab to close the flap, the two tabs, being of different colors so as to prevent the user from contaminating his hands.

5. The portable hand sanitizing system of claim 1 wherein a backside portion of the flap having a scrubbing surface to clean fingernails.

6. The portable hand sanitizing system of claim 1 wherein hook and loop patches are used for securing the flaps in either a closed or open configuration.

7. The portable hand sanitizing system of claim 1 wherein the fanny pack has belt loop and thigh straps to attach to a user.

8. The portable hand sanitizing system of claim 1 wherein the fanny pack has a ring, attached to the fanny pack for carrying a dry towel.

9. The portable hand sanitizing system of claim 1 having a method of use consisting in the steps of:
a) a user first filling the pockets with the sanitizing substance;
b) the user fills the pockets slowly to ensure that the absorbent lining material absorbs the sanitizing substance and that the liquid does not spill out of the pockets;
c) the user pulls with his finger on a pull out tab which is color coded to open a flap which exposes one of the two pockets. A backside portion of the flap having a scrubbing surface to clean fingernails;
d) the user then puts his hand inside the pocket which is moist with the sanitizing substance which is held captive by way of the absorbent lining material;
e) after use, the user grasps a pull back tab to close the flap, the pull tab and the pull back tab, being of different colors, preventing the user from contaminating his hands by having the user not touching the pull out tab after having washed his hands;
f) the user then repeats the process with his other hand, using the other pocket.
